(19) United States
(12) Patent Application Publication
(10) Pub. No.: US 2024/0189617 A1
(43) Pub. Date: Jun. 13, 2024

(54) LIGHT CAPSULE

(71) Applicant: Brian Michael Coyle, Canyon, CA (US)

(72) Inventors: BRIAN MICHAEL COYLE, Canyon, CA (US); Alys Larsen, Canyon, CA (US)

(21) Appl. No.: 18/241,822

(22) Filed: Sep. 1, 2023

Related U.S. Application Data
(63) Continuation-in-part of application No. 16/729,399, filed on Dec. 29, 2019, now Pat. No. 11,800,851.

(51) Int. Cl. A61N 5/06; H01M 8/16; H02N 1/04

(52) U.S. Cl. CPC A61N 5/0603; A61N 5/0624; H01M 8/16; H02N 1/04; A61N 2005/0609; A61N 2005/0626; A61N 2005/0632; A61N 2005/0643

(57) ABSTRACT

The present invention relates to devices, methods, and systems that emit light in the ruminant reticulorumen, to inactivate a distinct group of microbes. The device size and weight obtains a predetermined position in the rumen, and protectively contains a first power source, electric components, and light sources. The present invention contains methods of duty cycles and systems of second power sources to enable the first power source to operate the light sources for a prolonged period.

### RELATED APPLICATIONS

This present application is a continuation-in-part of co-pending U.S. application Ser. No. 16/729,399 filed Dec. 29, 2019, and entitled “LIGHT CAPSULE”, the entire disclosure of which is expressly incorporated herein by reference.

### STATEMENT REGARDING FEDERALLY SPONSORED RESEARCH OR DEVELOPMENT

Not Applicable

### FIELD OF THE EMBODIMENTS

The present invention relates to systems and methods for inactivating a distinct group of microbes in the reticulorumen of ruminants with light for a prolonged time using power source controls and second power sources.

### BACKGROUND OF THE INVENTION

This invention relates generally to an encapsulated luminaire designed to be deposited in a stomach of a ruminant animal, such as a cow, for inactivating some of the bacteria in the animal's digestive system. More particularly, the invention pertains to a method of promoting animal growth and health that can substitute for the use of sub-therapeutic dosages of antibiotics, without inducing antimicrobial resistance, which has become a public health crisis.

The ruminant digestive system exploits bacteria. Vast numbers (hundreds of trillions) of fermentative bacteria flow into their stomachs. These bacteria digest foodstuffs and are themselves a major source of protein. The abomasum secretes large amounts of lysozymes, enzymes that hydrolyze the glycosidic bonds in bacterial cell wall mucopolysaccharides. Once ruptured, the bacterium's internal material is consumed by other bacteria, or absorbed by the animal. Bacterial residues make up a significant part of a ruminant's protein source.

This dual use of bacteria by ruminants establishes a competitive equilibrium. Higher bacteria levels can lead to higher levels of protein consumption, and increased fermentation can lead to more carbohydrate and energy consumption. But bacteria also compete with the animal for food, particularly when they are in a state of equilibrium. Killing some bacteria with interventions like antibiotics pushes bacteria below the equilibrium, increasing the exponential growth phase optimal for protein production.

When a ruminant eats, particles flow down the esophagus and into the rumen, where they flow freely between the rumen and reticulum, hence the term reticulorumen. This area contains about half the digestive capacity, in a full grown cow perhaps 40 liters of an 80 liter digestive capacity. It contains the majority of microbial activity. The rumen contracts severely to mix its contents. Most animals cannot digest cellulose. Ruminants delegate the process to microbes. These digest cellulose to produce their own energy, and release volatile fatty acids that provide 70% of the cow or steer's energy supply. Microorganisms also produce prodigious amounts of CO₂ and methane.

The lower layers of bovine reticulorumen are ideal for organisms that can't compete with oxygen-users but need their by-products. These include methanogens. The enteric methane they produce and which animals eruct (belch) generates 5% of global warming gasses. Various methods have been proposed to control methanogen activity in ruminant reticulorumen. Enteric methane controls include vaccines and antibiotics, which are expensive and resistance prone, and exotic chemicals and foodstuffs whose effect, production and availability on a global scale are doubtful and also costly as they are used continuously.

It has been shown that 420 nm light kills methanogens. It has be shown that 405 nm light kills bacteria. 405 nm light is promoted by the U.S. Food and Drug Administration for use in blood transport, to prevent pathogen transmission without use of antibiotics.

U.S. application Ser. No. 16/729,399, of which this is a continuation in part, describes bolus-shaped light capsules that contain light sources, emitting wavelengths that inactivate some microbes. These light capsules are shot into cows with a bolus gun, and settle in the reticulorumen anaerobic fraction layer where methanogens concentrate. The light capsule light sources emit specific wavelengths that inactivate distinct groups of microbes. The light capsule is of sufficient weight and size to be retained in the rumen for the life of the animal. It contains a battery which can operate the light sources for a prolonged period, defined as multi-month to multi-year.

Prior art does not describe detailed mechanisms to extend battery life. These are needed, particularly for dairy cows, whose lifespans are commonly at least five years. There are 10 million dairy cows in the United States, 23 million dairy cows in Europe, 25 million dairy cows in Brazil, and 300 million dairy cows in India.

### SUMMARY OF THE INVENTION

The present invention provides a bolus-shaped light capsule that contains light sources that inactivate some microbes. In its basic form the invention comprises a light emitting assembly adapted to be located in the reticulorumen of a ruminant animal. In embodiments described herein, the luminaire power sources are operated to maximize power source lifespan and treatment effect. Some embodiments are recharged by the reticulorumen environment. The methods, systems and apparatus include the generation of electricity to drive the light sources in the bolus-shaped light capsule as it is retained in the reticulorumen of an animal.

The light capsule may emit microbe inactivating light continuously, periodically, at predetermined times, determined by one or more of: sensor-detected local environmental conditions, instructions of an external operator, predetermined energy management settings, reprogrammed energy management settings, feedback-driven energy management systems, and other energy management methods that extend the life of a power source. A reduction in emitted light may be caused by fouling of light apertures. These may be cleaned with ultrasonic vibration, from a transducer vibrator embedded or enclosed near the emitting light.

The light capsule provides a first power sources sufficient to operate the light sources for an extended, multi-year period, a controller that manages electrical power, and thermal management. In some embodiments the controller includes Pulse Width Modification (PWM) to pulse device light sources on and off, on a schedule that generates sufficient light energy to inactivate specific microbes while preserving available power sources. This is called a duty cycle, and pulses may be emitted at a Nyquist rate for specific microbes, the frequency that inactivates the specific microbes. Some microbes have subpicosecond electron transfer dynamics, which require small energetic driving forces. In an embodiment the PWM will emit light for 1 millionth of a second out of every 10 millionths seconds, or 9 MHz OFF, 1 MHz ON. This is a 0.1 periodic operation, and extends power source operation 10 times, minus conduction, switching, and gate loss. Other embodiments use duty cycles with longer or shorter ON and OFF periods, including PWM that flexibly modify these periods in response to regeneration power provided by a second power source.

A further embodiment of the light capsule employs a Triboelectric Generator (TG) as the second power source to supply regeneration power to the first power source. TGs use contact electrification of two materials with different electro-polarization capabilities caused by kinetic energy of a movable system. Piezoelectric TGs generate electric recharge energy through the mechanical deformation of piezoelectric materials; they harvest small mechanical energy. Electromagnetic TGs are larger. Cattle rumen cause large mass displacements, with more kinetic energy potential than human activity. To harvest relatively large kinetic energy found in cattle rumen, an embodiment uses electromagnetic TGs with low damping values. This causes greater displacements during oscillations, which harvests energy >500 μW. An embodiment uses Schottky TGs whose components remain in contact during energy harvesting and generate 3-6 Volts with sufficient kinetic movement.

Within a cow's rumen, a cycle of contractions occurs 1 to 3 times per minute. Primary contractions start in the reticulum and pass towards the rear of the rumen then circle forwards. A contraction wave is followed by a relaxation wave. Part of the rumen contracts while other parts dilate, which in an embodiment provide movement transferred to a TG. Secondary contractions are associated with eructation (belching.) Although less frequent than primary contractions, eructed gas can travel up the esophagus at 160 to 225 cm per second (around 5 mph). On average, about 30-50 liters of gas is belched by an adult cattle per hour; this causes significant tissue movement, which in an embodiment is transferred to a TG.

Although a light capsule's density and size cause it to remain in a certain location of the cow's rumen, it is jostled and rocked by the energetic forces around it. In an embodiment, this constant stimulus generates electrical power through the TG. In an embodiment a TG is housed inside the light capsule body. In another embodiment a TG is housed in a container that is outside the light capsule, with a conduit tube connecting it to the light capsule. In a preferred embodiment, a voltage conditioner regulates the discharge power from the TG. This transforms TG output to an electronically useful voltage, which is then used to recharge a capacitor. The capacitor is replenished by the TG during rumenoreticulum contractions. The voltage conditioner maximizes output power, transforming voltage of output power sufficient to operate a plurality of high-power LEDs.

In one embodiment a TG produces ˜1 mW at a current of ˜1.5 mA with ˜200 V resistance, for maximum power and current density of about 110 mW/m2 and 175 mA/m2, and a stable power density of ˜50 mW/m2 (0.96 V). In another embodiment a stable power density of ˜1.75 V is obtained. Other embodiments achieve different stable power densities. The reticulorumen environment maintains daily power generation. Further embodiments include a system to store and accumulate power. In tandem with the controller, the TG system may operate 4 LEDs of 500 mA with luminous flux at a sufficient forward current to inactivate targeted microbes over a prolonged period.

Another embodiment uses a microbial fuel cell (MFC) to generate electricity using the metabolic energy of an animal's digestive microbes, which use cellulose as their food and produce energy. Ruminal microbes hydrolyze cellulose anaerobically, and are electrochemically active. These microbes include metabolically active species that have high power densities. Rumen microbes maintain constant power generation without exogenous electron transfer mediators, for as long as cellulose is available. The diversity of these microbes, in the tens of thousands of species, are needed to digest the very great number of metabolic intermediates produced in cellulose degradation. A fuel cell has an anode and a cathode. Microbes oxidize the anode and electrons flow to the cathode, which is electrically coupled to the anode but separated by a membrane. The cathode needs an electron acceptor, usually oxygen because of its high redox potential. However there is almost no dissolved or gaseous oxygen in a cow reticulorumen. An embodiment uses a microbial fuel cell that employs an alternative oxide as the cathode reactant, instead of oxygen. A preferred embodiment uses manganese oxides. A matrix formed of a carbon fibers with manganese oxide centers provides cathode reaction sites and an electron transport network. Electron movements are generated by strong electrostatic attraction towards manganese oxide cations. Electrons flow spontaneously through the membrane to an external circuit, creating a flow of electricity. The manganese ions dissolved from manganese oxide reversibly deposit back to the cathode and preserve the manganese oxides for energy storage. In an embodiment manganese-oxidizing prokaryotes produce the cathode reactant.

Further aspects, elements and advantages of the present invention will be understood by those of skill in the art upon reading of the description set forth below.

### BRIEF DESCRIPTION OF THE DRAWINGS

FIG. 1A is a graph of a baseline duty cycle for a light capsule of the present invention.

FIG. 1B is a graph of a duty cycle configured to extend power source capacity.

FIG. 1C is a graph of a duty cycle configured for specific microbial inactivation r.

FIG. 2A is a circuit diagram integrating a second regenerative power source.

FIG. 2B is a graph illustrating a PWM integrating a second a regenerative power source.

FIG. 3 illustrates a second regenerative power source that is an internal TG and TG components.

FIG. 4 illustrates a second regenerative power source that is an external TG and TG components and circuit diagram.

FIG. 5 illustrates a second regenerative power source that is an MFC and MFC components.

### DETAILED DESCRIPTION

The following detailed description and the accompanying drawings are intended to describe some, but not necessarily all, examples of embodiments of the invention. The contents of this detailed description and the accompanying drawings do not limit the scope of the invention in any way.

While the invention has been described in multiple embodiments, the words which have been used are words of description rather than limitation. Changes may be made within the purview of the claims without departing from the scope and spirit of the invention. For example, any element or attribute of one embodiment or example may be incorporated into or used with another embodiment or example, unless otherwise indicated or unless doing so would render the embodiment or example unsuitable for its intended use. For example, materials may be used for membranes, electrodes, anodes, cathodes, and/or other components as may be sourced and utilized, other than the materials described herein. Also, each embodiment was shown as containing LEDs, but light sources of differing sizes and shapes may be used. The dimensions may be varied as appropriate to user needs and manufacturing specifications. All reasonable additions, deletions, modifications and alterations are to be considered equivalents of the described examples and embodiments are to be included within the scope of the following claims.

Controlling high brightness LEDs with PWM signals is a well-known and a widely implemented method of adjusting LED performance. The ‘ON’ part of the pulse train drives the LED at a forward voltage specification, preserving color temperature. A duty cycle will decrease the LED's luminosity, since fewer photons reach a destination. The switching frequency can be tailored to microbial electron absorbance, which is determined by frequency as well as luminosity. A preferred embodiment uses a PWM signal generator to extend power source lifetime. The circuitry includes a power source that delivers the desired current to the LED. The current is turned on and off by the PWM control signal. The instantaneous current supplied to the LED remains constant. Numerous microprocessors have PWM control hardware built in.

However research indicates that PWM duty cycles change the junction temperature of the LED; and junction temperature alters the color emitted by the chip. The light capsule emits specific wavelengths known to inactivate specific microbes. If duty cycles change the LED wavelength, they pose a problem. Researchers varied PWM from 3 to 100%, and measured wavelength output. Peak wavelengths moved toward shorter, bluer wavelengths as the PWM decreased. At the briefest PWM (ON 3%) wavelength shifted 2.5 nm towards blue.

An embodiment solves this problem. A light of specific wavelength must be emitted. It is determined that the light is operated at a PWM that is ON a specific percentage of time, between 0.1% and 99.9% of the time. Depending on the percent of time ON of the embodiment, the emitted wavelength is adjusted to be longer or shorter, in the opposite direction of the change due to the impact of the junction temperature. The lower the duty cycle, the lower the junction temperature of the LED, which would cause shorter wavelengths and bluer light, but the adjusted wavelength emitted is longer by the same amount.

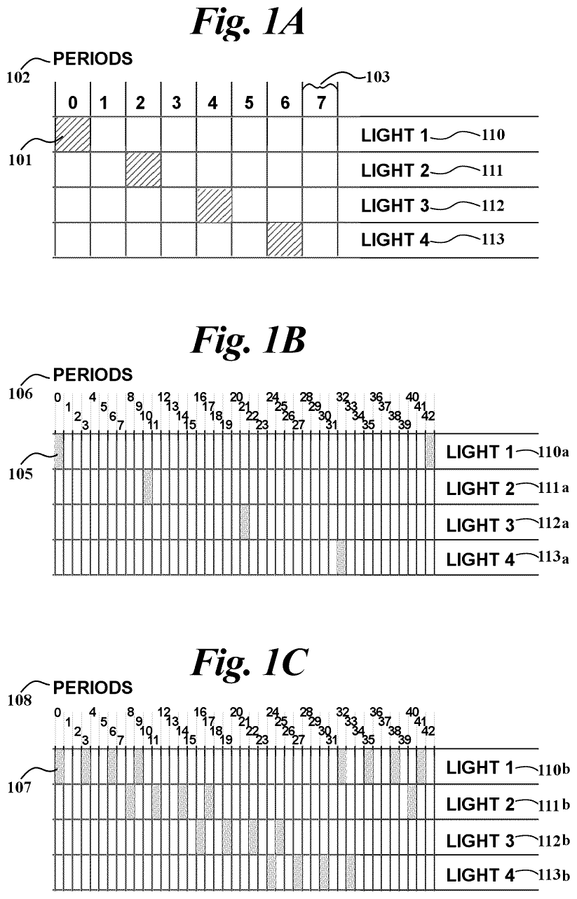

FIGS. 1A, 1B, and 1C illustrate PWM duty cycle embodiments for multiple light sources. Such a system prevents overuse of power, extends power source life, and can be easily modified when energy sources fluctuate. FIG. 1A shows a baseline PWM, wherein each light source, such as 110, 111, 112, and 113, is energized briefly in succession for one of a plurality of periods 102, and the system is ON 50% of the time. Hatched areas, such as 101, represent the ON time period. In this embodiment any time period, such as 103, is equivalent in measured time as other time periods. FIG. 1B shows a PWM configured to further extend power source capacity; each light source, such as 110a, 111a, 112a, and 113a, is energized briefly in succession for one of a plurality of periods 106, and the system is on 10% of the time. Hatched areas, such as 105, represent the ON time period. FIG. 1C shows a PWM configured for specific microbial inactivation, which are periods 108. Hatched areas, such as 107, represent the ON time period. The critical pulse train is emitted by each light source, such as a first light source 110b, a second light source, such as 111b, a third light source, such as 112b, a fourth light source, such as 113b, and so on. This embodiment may include light sources emitting light pulses that overlap each other in time. Although FIGS. 1A, 1B, and 1C illustrate embodiments with four light sources, embodiments using duty cycles may operate any number of light sources at any PWM. Also embodiments may include configurations where each light source has its own specific PWM. Embodiments include PWM that dynamically change.

The light capsule may have a second power source with a regenerative capacity, in which energy is recovered from kinetic motion in the rumen, or from microbial activity, or from external inputs such as from a radio frequency emitter. In these cases the external inputs may not be used directly to effectively power the light capsule without a control interface. In accordance with the present invention, an embodiment with one or more second regenerative energy sources is connected to one or more first power sources in the light capsule. Because second regenerative power sources may not permit continuous light capsule operation, a duty cycle embodiment resolves the mismatch between input power and operations. An embodiment with a second regenerative power source uses voltage measurements, rather than predetermined cycles, to determine the duty cycle. In this case the ON period when the resistor is connected ends when voltage decreases below a predetermined threshold.

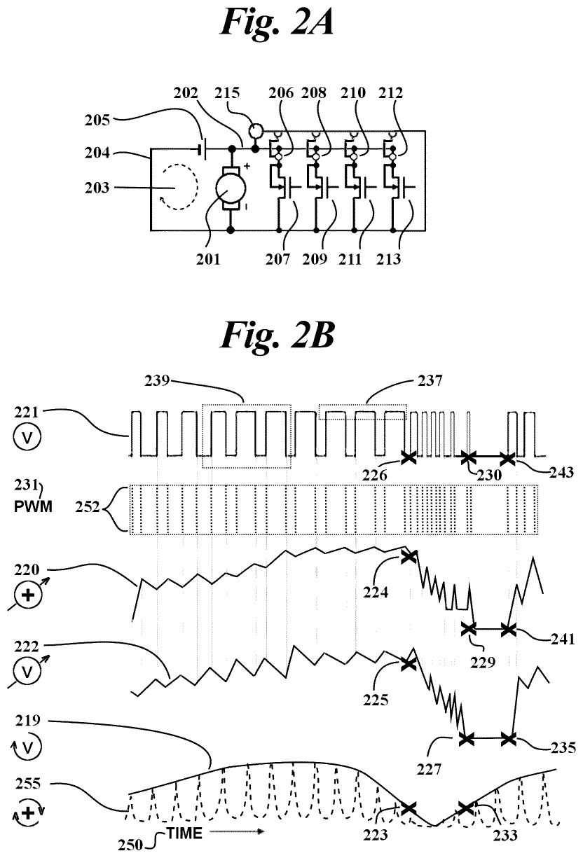

FIGS. 2A and 2B illustrate an embodiment in which a second regenerative power source is employed. In FIG. 2A the first power source 201 provides the first voltage U 202. The second voltage source is a regenerative energy source 203 that provides the second voltage R 204, which enters the system at regenerative energy terminal 205, which presents low pass circuitry and impedance that filters R 204, and also can disconnect R 204. U 202 is applied to light sources 207, 209, 211, and 213 through a controller 215 that executes one or more PWM via switches 206, 208, 210, and 212.

FIG. 2B illustrates the current generated in an embodiment with two voltage sources, a second regenerative power source and a first power source, showing how a variable current 220 is integrated by a PWM 231. This embodiment has a PWM 231 that produces an adjustable duty cycle shown within the dotted lines 252 that generates an applied voltage 221 by calculating a variable current 220 over time 250. In an embodiment where the second regenerative power source produces a sinusoidal energy output, as illustrated in dotted wave function 255, a rectifier generates a direct current regenerative voltage R 219. The adjustable duty cycle 252 results from the PWM integration of regenerative voltage R 219 with first power source voltage U 222. If R 219 decreases below a first predetermined minimum voltage threshold 223 then the variable current 220 at 224 is drawn only from U 222 at 225. The PWM 231 adjusts the applied voltage 221 at 226 to minimize the variable current 220 at 225. If U 222 decreases below a second predetermined minimum threshold 227, U 222 is disconnected from a load and the variable current 220 stops at 229 and applied voltage 221 ceases at 230. If R 219 increases above the first predetermined minimum voltage at 233, it recharges the first power source voltage U 222. When U 222 is recharged above the second predetermined minimum voltage at 235 it is reconnected to the load and the variable current 220 flows at 241 to applied voltage 221 at 243. The increase in R 219 that recharges U 222 is measured by the PWM 231, which integrates the variable current 220 by modifying the PWM 231 adjustable duty cycle 252, so that when ON the applied voltage 221 always provides a constant voltage to a load, such as at 237. The illustrated embodiment shows that ON periods such as 239 extend when the variable current 220 is increased, however other embodiments use other PWM pulse rates. In an embodiment the variable current 220 increases and decreases as energy is generated and used. The PWM 231 period may change when R 219 increases, and shrink when R 219 decreases, to provide the constant current to the load.

Resistance mismatch between the second regenerative power source and the electrical load can be resolved through periodic connection and disconnection of the load at a relatively high frequency, defined as 0.05 to 50 Hz.

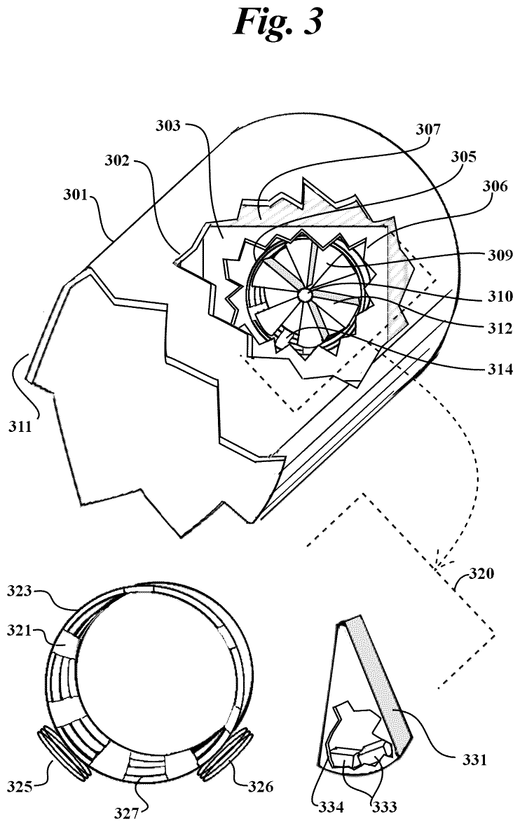

FIG. 3 illustrates an embodiment in which a TG 303 is employed, with TG components 305 visible through a cutaway 306 of the TG container 307, which itself is visible through a cutaway 302 of a light capsule 301, which at 311 is cutoff from full size. This is an embodiment where the TG 303 is within the light capsule 301. In an embodiment the TG components 305 are an electromagnetic generating swing-structure 309 composed of rotors 312 coupled to an axis 310 that transmits motion from a rumen causing the rotors 312 to swing over electrodes 314, and electrons pass in the opposite direction. A further embodiment includes the coupling of triboelectrification and electrostatic induction or other hybrid process. Rumen contractions have periods of a second or more, and may have superimposed vibrations. Embodiments with swing and pendulum structures are used for such energy sources. Using such structures extends operation time, multiplies frequency, with high stability current and voltage pulse. At 320 some TG components are enlarged. Electrodes, such as electrode 321, are embedded in a shell 323 with additional copper coils 325 and 326 attached on the outside of the shell 323. An embodiment uses copper coils 325 and 326 because they intensify induced current. Another embodiment increases current levels by employing frictional material 327 on the inside of shell 323. A single rotor 331 is shown enlarged, with magnets 333 exposed in a cutout 334 embedded at the bottom of the rotor 331 to generate electromagnetic flux.

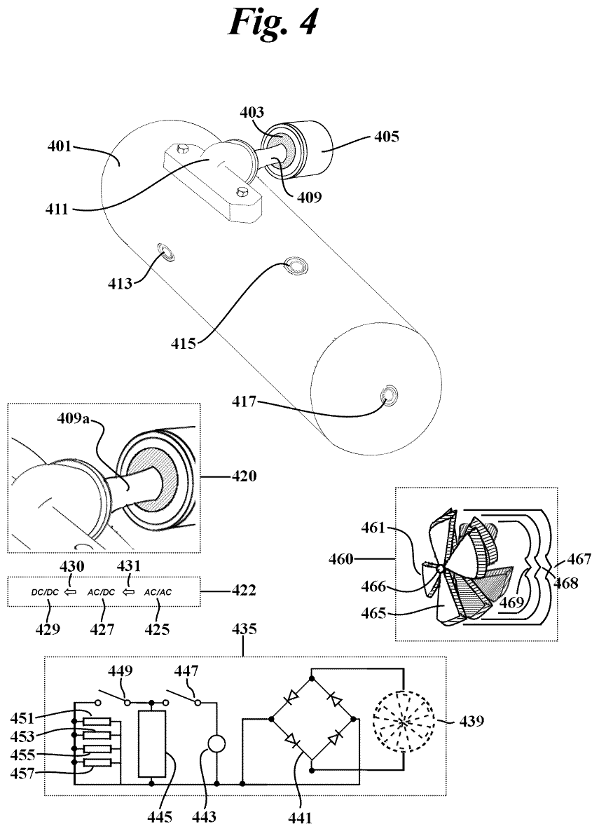

FIG. 4 illustrates an embodiment in which a TG 403 is employed, housed in a separate unit 405 which affords the TG 403 a greater opportunity to be buffeted by rumen contractions. The separate unit 405 is connected to the light capsule 401 with a conduit tube 409 which contains a bridge rectification component of a Power Management System (PMS), and is flexible to afford the separate unit 405 mobility. The PMS is a voltage conditioner. Because electron transfer operations need to maximize efficiency, the cathode in the light capsule 401 is mounted in a housing 411, close to the TG 403. On the light capsule 401 surface are apertures 413, 415, and 417, through which light passes. The conduit tube 409a is illustrated in an enlarged view 420, and at 422 is shown three modules 425, 427, and 429 that are inside of 409a and comprise the PMS bridge rectification component. In an embodiment the three modules 425, 427, and 429 are in a series from the TG to the cathode area, the direction illustrated by arrows 430 and 431. The modules include an alternating current to alternating current (AC/AC) conversion module 425, an alternating current to direct current (AC/DC) conversion module 427, and a direct current to direct current (DC/DC) conversion module 429. A circuit diagram 435 illustrates an embodiment with a PMS, where a regenerative power source 439 charges a capacitor 443 through a bridge rectification component 441. Once voltage reaches an impedance match condition, energy transfers from the capacitor 443 to the power storage unit 445. A switch 447 opens to avoid interference of the capacitor charging process. A switch 449 opens when the power storage unit 445 voltage falls below a threshold. Above the threshold, switch 449 closes and power is delivered to a load comprising four light emitting units 451, 453, 455, and 457, however in other embodiments different numbers of light emitting units are used, including embodiments that use a single light emitting unit, as well as those that have a plurality of greater than four. Enlarged view 460 shows an embodiment that uses a plurality of swing layers in a swing-structure regenerative power source 461, as may be contained in the separate unit 405, with a plurality of rotors, such as rotor 465, that radiate from a center hub 466 to form a first swing layer 467, wherein the swing-structure 461 contains a second swing layer 468 and a third swing layer 469 behind the first swing layer 467.

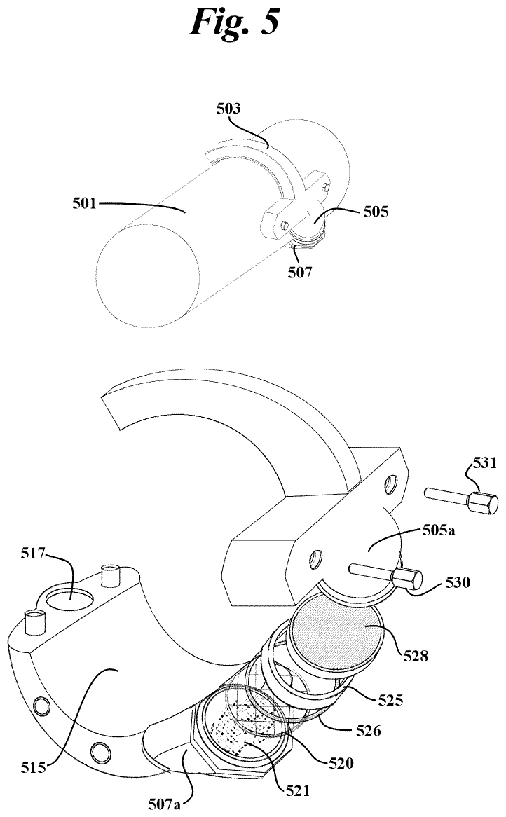

In a further embodiment, the regenerative power system is a microbial fuel cell (MFC). FIG. 5 illustrates a two-chamber MFC 503 as mounted on a light capsule 501; the MFC is composed of a cathode portion 505 and an anode portion 507, which are further shown as cathode portion 505a and anode portion 507a in a disconnected state that is enlarged. In an embodiment the anode portion 507a includes a microbial supply container 515 that is in fluid communication with rumen contents through portal 517 and is connected to the anode portion 507a; an electrolyte membrane 520 sufficient to separate the anode portion and the cathode portion; a brush substrate of the anode 521 is proximate to the electrolyte membrane; a gasket 525 and O-ring 526 connects the anode portion and the cathode portion. In an embodiment the brush substrate 521 is a biocompatible carbon based material. In a preferred embodiment the cathode portion 505a contains biomineralized manganese oxides in a matrix 528 as the cathodic reactant. Because reticulorumen have very limited oxygen available, in this embodiment the cathode does not use dissolved oxygen as a cathode reactant. In a further embodiment manganese-oxidizing prokaryotes produce the cathode reactant. In an embodiment the anode portion 505a is held in place by screws 530 and 531. In the MFC embodiments, microbes have a constant supply of food used as fuel by ruminant microorganisms in the reticulorumen.

While the invention has been described in multiple embodiments, the words which have been used are words of description rather than limitation. Changes may be made within the purview of the claims without departing from the scope and spirit of the invention. For example, any element or attribute of one embodiment or example may be incorporated into or used with another embodiment or example, unless otherwise indicated or unless doing so would render the embodiment or example unsuitable for its intended use. For example, materials may be used for membranes, electrodes, anodes, cathodes, and/or other components as may be sourced and utilized, other than the materials described herein. Also, each embodiment was shown as containing LEDs, but light sources of differing sizes and shapes may be used. The dimensions may be varied as appropriate to user needs and manufacturing specifications. All reasonable additions, deletions, modifications and alterations are to be considered equivalents of the described examples and embodiments are to be included within the scope of the following claims.

1. A light capsule retained in a ruminant reticulorumen, including
a) at least one light source emitting a radiation of at least one wavelength that inactivates at least one distinct group of microbes;
b) the light capsule having a weight to permanently position the light capsule in at least one layer of the reticulorumen;
c) a power source;
d) circuit elements to deliver power in at least one duty cycle to operate the at least one light source for a prolonged period.

2. The light capsule according to claim 1, wherein a change is prevented, the change being caused by the at least one duty cycle to the at least one wavelength that inactivates the at least one distinct group of microbes;
comprising the at least one light source has the at least one wavelength modified to offset the change caused by the at least one duty cycle.

3. The light capsule according to claim 1, wherein the duty cycle has at least one pulse width that increases the inactivation of the at least one distinct group of microbes.

4. The light capsule according to claim 1, wherein a sensor modifies at least one pulse width of the duty cycle.

5. The light capsule according to claim 1, wherein a communication modifies at least one pulse width of the duty cycle.

6. A light capsule retained in a ruminant reticulorumen, including
a) at least one light source emitting a radiation of at least one wavelength that inactivates at least one distinct group of microbes;
b) the light capsule having a weight to permanently position the light capsule in at least one layer of the reticulorumen;
c) a first power source within the light capsule;
d) a second regenerative power source;
e) a bridge circuit for performing power regeneration;
f) a rectifying circuit to supply the regeneration power to the first power source;
g) circuit elements to deliver power to the at least one light source in at least one duty cycle;
h) a controller that couples the second regenerative power source, regeneration power, the first power source, circuits, and the at least one duty cycle.

7. The light capsule according to claim 6, wherein the controller adjusts a pulse width of the at least one duty cycle.

8. The light capsule according to claim 6, wherein a resistance mismatch between the second regenerative power source and the at least one light source is resolved through periodic connection and disconnection of the circuit elements at a frequency of 0.05 to 50 Hz.

9. The light capsule according to claim 6, wherein the second regenerative power source is a triboelectric generator;
wherein the triboelectric generator is configured to harvest kinetic energy from a movement in the reticulorumen.

10. The light capsule according to claim 9, wherein the triboelectric generator is inside the light capsule.

11. The light capsule according to claim 9, wherein the triboelectric generator is outside of the light capsule.

12. The light capsule according to claim 6, wherein the second regenerative power source is a triboelectric generator that comprises at least one rotor coupled to an axis that transmits motion from the reticulorumen;
wherein a movement from the reticulorumen allows the at least one rotor to generate a triboelectric energy.

13. The light capsule according to claim 6, wherein the second regenerative power source is a microbial fuel cell that obtains electrons from microbial redox processes in the reticulorumen.

14. The light capsule according to claim 13, wherein the microbial fuel cell includes:
a) an anode container comprising an anode and an anode catalyst;
b) a population of microbes in contact with the anode,
wherein the population of microbes catalyze oxidation of a material in the reticulorumen;
c) a cathode compartment comprising a cathode and a cathode catalyst,
wherein the cathode is electrically coupled to the anode;
the cathode catalyst catalyzing a reduction reaction;
d) a membrane positioned between the anode compartment and the cathode compartment;
wherein a flow of electrons passes through the membrane to a circuit element.

15. The light capsule according to claim 13, in which the cathode contains manganese oxides.

16. The light capsule according to claim 6, wherein the second regenerative power source is a power source outside of the ruminant;
wherein the regeneration power is transmitted via an ultrasonic vibration through the ruminant body to a receiver in the light capsule.

17. A method for inactivating a distinct group of microbes in a reticulorumen for a prolonged time, comprising:
establishing, by a power controller of a light capsule positioned in the reticulorumen, a circuit between an onboard power source and a load in the light capsule;
controlling, by the power controller, a supply of a stable current to the load in order to activate at least one light source that emits wavelengths that inactivate the distinct group of microbes;
controlling, by the power controller, a duty cycle process in order to extend a lifespan of the onboard power source for the prolonged time.

18. The method of claim 17, wherein the power controller supplies a charging current from a regenerative power source to a component conditioning the charging current for the onboard power source.
